(19) United States
(12) Patent Application Publication
(10) Pub. No.: US 2024/0240227 A1
(43) Pub. Date: Jul. 18, 2024

(54) AUTOMATED MICROBIOLOGICAL LABORATORY FOR QUANTITATIVE AND QUALITATIVE MICROBIOLOGY

(71) Applicant: Institute for Environmental Health, Inc., Lake Forest Park, WA (US)

(72) Inventor: Mansour Samadpour, Lake Forest Park, WA (US)

(21) Appl. No.: 18/414,396

(22) Filed: Jan. 16, 2024

Related U.S. Application Data
(60) Provisional application No. 63/439,562, filed on Jan. 17, 2023.

(51) Int. Cl. C12Q 1/22; C12Q 1/06

(52) U.S. Cl. CPC C12Q 1/22; C12Q 1/06

(57) ABSTRACT

Provided are integrated automated systems for quantitative and/or qualitative microbiological assessment and/or analyte assessment of samples using in situ generated culture devices. The systems comprise: an integrated automated system comprising sterile media and/or buffer reagents, culture device parts, and automated components for media and/or buffer handling, test sample handling, culture device assembly, and microbial enumeration and/or detection, all within a sterile environment; and one or more processors; and memory, including computer-executable instructions that, if executed by the one or more processors, cause the integrated automated system to determine that a coded test sample, loaded into the system, is to be quantitatively and/or qualitatively assessed, and accordingly process and microbiologically assess the coded test sample using, for quantitative assessment, one or more specified in situ generated culture devices for quantitative assessment, thereby eliminating the need to sterilize, ship, store, and/or open ex situ fabricated culture devices prior to inoculation thereof.

### CROSS-REFERENCE TO RELATED APPLICATIONS

This application claims the benefit of U.S. Provisional Patent Application No. 63/439,562, filed Jan. 17, 2023, entitled “AUTOMATED MICROBIOLOGICAL LABORATORY FOR QUANTITATIVE AND QUALITATIVE MICROBIOLOGY,” the disclosure of which is incorporated herein by reference in its entirety.

### FIELD OF THE INVENTION

Aspects of the invention relate generally to integrated automatic systems that receive and optionally extract samples, prepare media and assemble culture devices in situ in a compact format, and inoculate the media/culture devices in situ, which may then be incubated and read to quantitatively determine the bioburden of the sample. In additional aspects, the systems also, or alternatively, provide qualitative sample assessment (e.g., pathogens, spoilage organisms, microbiome of the food, allergens, GMOs, toxins, ingredients, etc.) using, for example, genetic based or immunochemical based detection methods, etc.

### BACKGROUND

In microbiological testing of microbial bio-burden of samples, a portion of a sample is typically extracted and plated on various microbiological medias by pour plating, spread plating or spiral plating methods, each of which may allow one or more groups of organisms to grow to form colonies. The colonies, after appropriate incubation, are then counted, and the bio-burden is calculated and reported based on cfu/gram, or cfu./ml., or cfu/unit of a product or square centimeter/sq inch.

The microbiological plates most commonly used are petri dishes, but alternatives to petri dishes have been introduced into the market. For example, miniaturized alternatives include microfilm, petri-film, peel-plate, etc. These devices are manufactured centrally, sent to irradiation, and then distributed worldwide to laboratories.

A few companies also have developed automated systems where the extracted samples are loaded on the machines, and the machines automatically plate on various medias as required by the laboratory information management system (LIMS). While such automated systems greatly reduce labor cost, each automated system is designed for specific size and type of pre-assembled microbiological plates, the stock of which is influenced by supply chain and product shelf-life. While some of these systems make Petri dishes during the automated process, the assembled Petri dishes are problematic in a sense that they are bulky and require the addition of agar-based media at elevated temperature, typically resulting in solidification inside the transfer tubes. These systems also require water baths to maintain the media at elevated temperatures.

There is a need for automated systems with sample extraction capability, that have in-situ preparation of media and assembling and inoculation of compact culture devices (e.g., thin microfilms, Petri-pouch, etc.), which are integrated for efficiently and selectively visualizing and quantifying cultured microorganisms, and which avoid problematic clogging issues without hot media requirements, or the need for water baths.

There is a further need for such automated systems that additionally, or alternatively, provide qualitative laboratory tests including but not limited to testing for pathogens, spoilage organisms, microbiome of the food, allergens, GMOs, toxins, ingredients, etc., by performing analyte extractions followed by detection.

### SUMMARY OF EXEMPLARY ASPECTS OF THE INVENTION

Provided are integrated automatic systems that allow for an automated comprehensive quantitative and/or qualitative analysis of a samples. The automated system receives and extracts samples, produces media and assembles, in situ, culture devices in a compact format, and inoculates the media/culture devices, which may then be incubated and read to determine the bioburden of the sample, and/or qualitatively detect microorganisms in the sample.

The integrated and automated systems may provide in various portions or modules, for media mixing, dispensing, printing, sample extracting, diluting, dispensing and inoculation, in situ culture device assembly, incubation, image recognition and colony counting, and/or detection of target organisms, etc. Multiple versions of the in situ assembled culture devices may be used for quantification. In each case, the automated system will receive primary sample extracts in appropriate vessels with barcode issued by, e.g., a LIMS, the system will make appropriate sample dilutions, and an appropriate volume of each dilution is inoculated on the in situ assembled culture device(s).

Preferred versions of the integrated systems comprise integrated incubation and reader modules, and/or detection modules, that can automatically incubate the inoculated culture devices, and/or inoculated enrichment cultures, then read and/or detect, and report the data (e.g., to a LIMS system).

Embodiments of the disclosure can be described in view of the following clauses:

### An Automated Microbiological Laboratory for Quantitative and Qualitative Microbiology

1. A system for qualitative and/or quantitative microbiological assessment, comprising:
an integrated automated system comprising sterile media and/or buffer reagents, culture device parts, and automated components for media and/or buffer handling, test sample handling, culture device assembly, and microbial enumeration and/or detection, all within a sterile environment; and one or more processors; and memory, including computer-executable instructions (e.g., LIMS instructions) that, if executed by the one or more processors, cause the integrated automated system to determine that a coded test sample, loaded into the system, is to be quantitatively and/or qualitatively assessed, and
for quantitative assessment
   a) assemble, in situ, using the culture device parts, a specified type of microbiological culture device for culturing and quantitatively processing the coded test sample;
   b) inoculate the specified type of culture device, as part of (e.g., during or after) the in situ assembly, with an appropriate amount of the coded test sample, or an extraction and/or dilution thereof; and
   c) attribute the inoculated culture device to the coded test sample, wherein the assembling and inoculating are performed in situ by the integrated automated components of the system in the sterile environment, thereby eliminating the need to sterilize, ship, store, and/or open ex situ fabricated culture devices prior to inoculation thereof; and/or
for qualitative assessment,
   A) add an amount of a media or concentrated media into at least a portion of the coded test sample or extraction thereof, to provide a coded enrichment culture; and/or
   A1) dilute a portion of the coded test sample or extraction thereof by adding an amount of a suitable analyte extraction buffer.
2. The system of clause 1, wherein execution of the computer-executable instructions further causes the system to:
for quantitative assessment,
   d) direct the inoculated, attributed culture device for incubation at a temperature and for a time suitable to promote the growth of colonies corresponding to one or more target microorganisms;
   e) enumerate the colonies using the enumeration components in situ; and
   f) store and/or transmit the enumeration data attributed to the coded test sample; and/or
for qualitative assessment, execution of the computer-executable instructions further causes the system to:
   B) direct the enrichment culture for incubation in an incubator at a temperature and for a time suitable to promote the growth of one or more target microorganisms to reach detectable levels (and preferably become uniform throughout) to provide an enriched sample,
   C) detect, using at least an aliquot of the enriched sample, the one or more target microorganisms using the detection components in situ; and
   D) store and/or transmit the detection data attributed to the coded test sample for storage and/or reporting and/or analysis; and/or
   B1) direct the analyte sample of A1) to a suitable analyte extraction module/components for analyte extraction, using extraction components of the system;
   C1) detect, using a portion of the extracted analyte of B1) in a detection module/component of the system one of more extracted analytes; and
   D1) store and/or transmit the detection data of C1) attributed to the coded test sample for storage and/or reporting and/or analysis.
3. The system of clause 2, wherein directing, in d), the inoculated, attributed culture device for incubation, and/or directing, in B), the enrichment culture for incubation, comprises incubating in situ using an incubator integrated into the system, and/or incubating ex situ using a non-integrated incubator.
4. The system of clause 2 or 3, wherein:
enumerating, in e), the colonies in situ comprises using an integrated imaging device and image analysis program; and/or
detecting, in C), the one or more target organisms in situ comprises using a suitable detection assay in situ; and/or
detecting in C1), the one or more extracted analytes in situ comprises using a suitable detection assay in situ.
5. The system of clause 4, wherein the detection assay in C) is based on one or more selected from RNA, DNA, and immunochemistry; and/or
wherein the detection assay in C1) is based on one or more selected from ELISA and/or lateral flow.
6. The system of any one of clauses 1-5, wherein:
for quantitative assessment,
when the coded test sample loaded into the system is a primary sample, execution of the computer-executable instructions further causes the system to:
   mix or homogenize the coded test sample with a suitable amount of a specified buffer, and/or media, to provide an extracted sample;
   withdraw a subportion of the extracted sample; and
   dilute (e.g., serially and/or parallel, if so specified, the withdrawn portion to provide the extracted sample or the dilution(s) thereof for inoculation of the assembled culture device; and/or
when the coded test sample loaded into the system is an extracted sample, execution of the computer-executable instructions further causes the system to:
   withdraw a subportion of the extracted sample; and
   dilute (e.g., serially and/or parallel, if so specified, the withdrawn portion to provide the extracted sample or the dilution(s) thereof for inoculation of the assembled culture device; and/or
for qualitative assessment,
when the coded test sample loaded into the system is a primary sample, execution of the computer-executable instructions further causes the system to:
   prior to A), mix or homogenize the coded test sample with a suitable amount of a specified buffer to provide an extracted sample.
7. The system of any one of clauses 1-6, wherein the computer-executable instructions comprise one or more of LIMS instructions, PLC instructions, firmware, and programed instructions and logics.
8. The system of any one of clauses 1-6, wherein, for quantitative assessment, the specified type of in situ-assembled microbiological culture device comprises at least one selected from the group consisting of microfilm card, Petri-dish containing gel and or gum based media, and Petri-pouch.
9. The system of clause 8, wherein the in situ-assembled microbiological culture device is a Petri-dish, and wherein for the in situ assembly, execution of the computer-executable instructions further causes the system to:
remove the lid of a pre-loaded Petri dish having a base and a lid;
introduce an appropriate amount of a sterile mixture containing one or more medias plus one or more congealable polymers and/or gums;
introduce an appropriate amount of the coded test sample, or the extraction and/or the dilution thereof;
place the lid on the base of the Petri dish; and
mix the introduced sterile mixture and the sample, extraction or dilution thereof prior to gelation.
10. The system of clause 9, wherein the introducing of the sterile mixture and the introducing of the coded test sample, or the extraction and/or the dilution thereof is simultaneous.
11. The system of clause 9 or 10, wherein the one or more congealable polymers and/or gums of the introduced sterilized mixture does not congeal at ambient temperature in the absence of one or more cations, and wherein, prior to introducing the sterile mixture and the sample, extraction or dilution thereof, execution of the computer-executable instructions further causes the system to:
cover the base with a base gel layer containing the one or more cations, which are diffusible from the base gel layer into the introduced sterile mixture and the sample, extraction or dilution thereof.
12. The system of clause 11, wherein the one or more congealable polymers and/or gums of the introduced sterile mixture comprise pectin and/or alginate, and wherein the one or cations of the base gel layer comprises a divalent or trivalent cation.
13. The system of clauses 11 or 12, wherein the base gel layer comprises agar, gelatin, silica gel, or carrageenan.
14. The system of any one of clauses 11-13, wherein the one or cations comprises Ca²⁺.
15. The system of any one of clauses 9-14, wherein the sterile mixture is introduced in liquid form, or wherein the one or more congealable polymers and/or gums of the introduced sterile mixture is initially introduced as a coating in powder form, which upon the introduction of the media in liquid form and/or of the coded test sample, or the extraction and/or the dilution thereof, absorbs and forms a gel over the base gel layer.
16. The system of clause 8, wherein the in situ-assembled microbiological culture device is a microfilm device having a backing card and a top cover, and wherein for the in situ assembly, execution of the computer-executable instructions further causes the system to:
apply sterile media to the surface of the backing card;
introduce an appropriate amount of the coded test sample, or the extraction and/or the dilution thereof to be in contact with the media to form a mixture; and
place a top cover over the backing card; and
stamp the placed top cover to distribute the mixture over a desired test area of the backing card.
17. The system of clause 16, wherein the surface of the backing card defines a reservoir to receive the media and the coded test sample, or the extraction and/or the dilution thereof, the reservoir having a surface area defining the desired test area.
18. The system of clause 16 or 17, wherein, before or after applying sterile media to the surface of the backing card, execution of the computer-executable instructions further causes the system to:
apply an adhesive, gel, wax or grease in a pattern to define the desired area over which the mixture is distributed.
19. The system of clause 18, wherein the adhesive comprises a pressure sensitive glue.
20. The system of any one of clauses 15-19, wherein prior to placing the top cover, execution of the computer-executable instructions further causes the system to:
introduce an amount of concentrated media to be diluted by the introduced coded test sample, or the extraction and/or the dilution thereof.
21 The system of any one of clauses 18-20, wherein the adhesive, gel, wax or grease is applied prior to the sterile media.
22. The system of any one of clauses 16-21, wherein the applying of the sterile media to the surface of the backing card comprises:
printing the sterile media onto the surface of the backing card; and/or otherwise dispensing the sterile media onto the surface of the backing card followed by drying.
23. The system of any one of clauses 16-22, wherein the top cover is pre-coated, on a surface, with a gelling agent powder, with or without dry media or media components.
24. The system of clause 8, wherein the in situ-assembled microbiological culture device is a Petri pouch, having or configured to have an opening.
25. The system of clause 24, wherein for the in situ assembly, execution of the computer-executable instructions further causes the system to:
introduce, via the opening, an appropriate amount of a sterile mixture containing one or more medias plus one or more congealable polymers and/or gums;
introduce, via the opening, an appropriate amount of the coded test sample, or the extraction and/or the dilution thereof;
mix the introduced sterile mixture and the sample, extraction or dilution thereof within the pouch;
roll, press or otherwise form the pouch to distribute, prior to gelation, the mixed components to provide a desired gel thickness and surface area; and
close, prior to or after the mixing and/or the rolling, the opening.
26. The system of clause 24, wherein the sterile mixture is introduced in liquid form, or wherein the one or more medias and the one or more congealable polymers and/or gums of the introduced sterile mixture are initially introduced in powder form, which upon the introduction of the coded test sample, or the extraction and/or the dilution thereof, absorbs and forms a gel.
27. The system of clauses 25 and 26, wherein the one or more congealable polymers and/or gums of the introduced sterilize mixture does not congeal at ambient temperature in the absence of one or more cations, and wherein the inside of the Petri pouches is pretreated with the one or more cations, which are diffusible into the introduced sterile mixture and the sample, extraction or dilution thereof.
28. The system of clause 27, wherein prior to introducing the sterile mixture and the sample, extraction or dilution thereof, execution of the computer-executable instructions further causes the system to:
pretreat the inside of the Petri pouches with the one or more cations, which are subsequently diffusible into the introduced sterile mixture and the sample, extraction or dilution thereof.

### BRIEF DESCRIPTION OF THE DRAWINGS

Those of skill in the art will understand that the drawing(s), described below, are for illustrative purposes only. The drawings are not intended to limit the scope of the present teachings in any way.

FIG. 1 shows, by way of non-limiting examples of the present invention, pressure sensitive glue being dispensed in a ring and line pattern onto a backing card of a microfilm culture device at an assembly position.

FIG. 2 shows, by way of non-limiting examples of the present invention, sample and media being simultaneously dispensed into the test area of the microfilm backing card shown in FIG. 1.

FIG. 3 shows, by way of non-limiting examples of the present invention, stamping of a top film over the dispensed sample and media dispensed in FIG. 2.

FIG. 4 shows, by way of non-limiting examples of the present invention, addition, via a nozzle, of a sterilized base media (not shown) containing polymers that congeal in the presence of one or more of cations, gums, water, and appropriate salts containing the cations, etc., to cover the inner base surface of a Petri dish at an assembly position.

FIG. 5 shows, by way of non-limiting examples of the present invention, tilting, at an assembly position, of the Petri dish base of FIG. 4 at a sufficient angle and for a sufficient time to drain and pool excess (non-adhered) gel, and while tilted, an aspirator nozzle 408 aspirates the pooled gel, leaving a thin, formed gel coating (not shown) on the Petri dish base (a base gel layer).

FIG. 6 shows, by way of non-limiting examples of the present invention, multiple Petri dish bases containing adhered base gel layers stacked on an integrated carousel for rapid cooling.

FIG. 7 shows, by way of non-limiting examples of the present invention, a Petri dish base at an assembly position where an appropriate amount of a sample/dilution is added simultaneously with addition of an appropriate amount of a sterile mixture containing one or more medias plus one or more congealable polymers.

FIG. 8 shows, by way of non-limiting examples of the present invention, a Petri pouch at an assembly position, where the pouch is opened, allowing for introduction, via the opening, of a powder or liquid/paste media containing a gelling agent, and introduction of a sample/dilution for inoculation.

FIG. 9 shows, by way of non-limiting examples of the present invention, the petri pouch of FIG. 8, after gently being mixed and labeled, being passed through a roller with an appropriate gap to distribute the liquid sample and media within the pouch, to provide for subsequent gelation with an appropriate gel thickness.

FIG. 10 shows, by way of non-limiting examples of the present invention, a flow diagram showing implementation of computer-executable instructions for sample processing and quantitative analysis within an automated, integrated laboratory system for quantitative assessment of microbiological and other samples.

FIG. 11 shows, by way of non-limiting examples of the present invention, a flow diagram showing implementation of computer-executable instructions for sample processing and quantitative and/or qualitative analysis within an automated, integrated laboratory system for quantitative and/or qualitative assessment of microbiological and other samples.

### DETAILED DESCRIPTION OF THE INVENTION

Aspects of the invention overcome deficiencies of current methods for microbiological testing.

Provided is a process and integrated automated system for a quantitative and/or qualitative microbiological testing system with in-situ production of microbiological culture devices (e.g., Petri dishes, microfilms, Petri pouches) for microbiological culture. The integrated automated system receives primary samples having sample IDs, in appropriate containers, and/or extracted samples, having sample IDs, as a starting point. After reading the sample ID, the system communicates with e.g., the Laboratory Information Management System (LIMS), and receives testing instructions comprising dilution instructions, etc., and, in the case of quantitative assessment, instructions for plating of each dilution with one or more different media and with one or more different culture devices, based on the tests needed, and/or in the case of qualitative assessment instructions for inoculating enrichment cultures for target organism detection.

For extracting samples in automated fashion, each primary sample is extracted by introduction of an appropriate buffer or media, mixing, and if needed withdrawal of sub-samples for serial dilutions.

Microfilms (thin-films). A first exemplary culture device embodiment comprises is situ assembled microfilms. For quantitative assessment using such microfilms, for example, vessels containing extracted samples, or dilution vessels, are then moved into another position (plating position) of the automated system where an appropriate amount of each sample/dilution is plated on a respective micro-film culture device that is manufactured in situ in a separate module (thin-film assembly module) of the same automated system. For example, based on the LIMS instruction(s), a bottom card is picked up and placed in a microfilm assembly position. The card may consist of a piece of cardboard, or other suitable material, which has (e.g., is coated by, or otherwise comprises) a water impermeable barrier on at least one side.

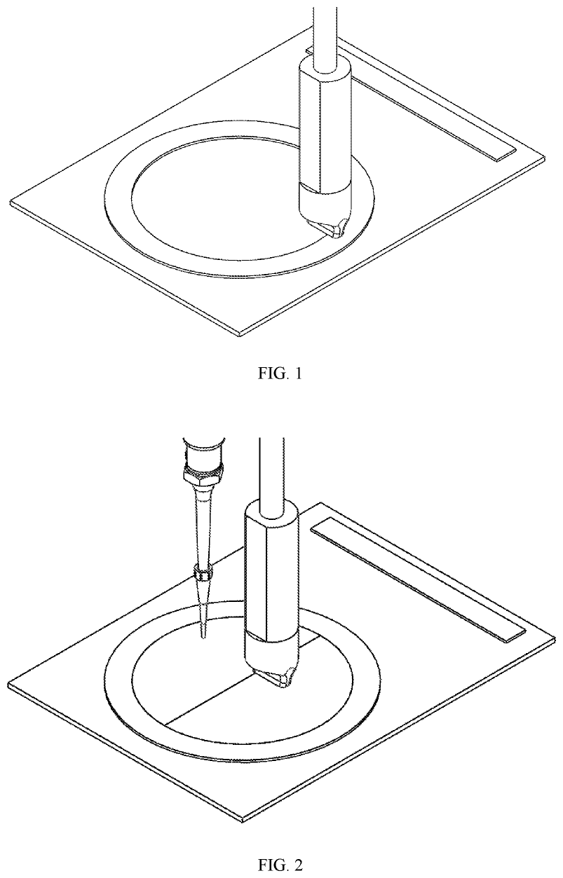

With reference to FIG. 1, the card 102 with the water impermeable barrier side 104 on top (e.g., facing upwardly) is moved into an assembly position 100 where, in preferred aspects, a layer of e.g., adhesive (e.g., glue), gel, wax, or grease, etc. may be deposited, via nozzle 106, in a shape (e.g., geometrical shape) defining the outer borders of a shape (test area) that will contain the media. FIG. 1 shows a pressure sensitive glue being dispensed as a ring 108 defining the test area 110, and as an optional separate line (ribbon) 112, on the bottom card (backing card) 102 for sealing purposes. Media may then deposited (e.g., printed) on the card (within the borders/test area 110 defined by the adhesive/glue/gel/wax/grease). In alternate embodiments, media may be deposited without a deposited defining boarder, or before depositing the defining border. After media deposition, or simultaneously therewith, an appropriate amount of the sample or sample dilution is added to the card 102, where the media is deposited or is being deposited.

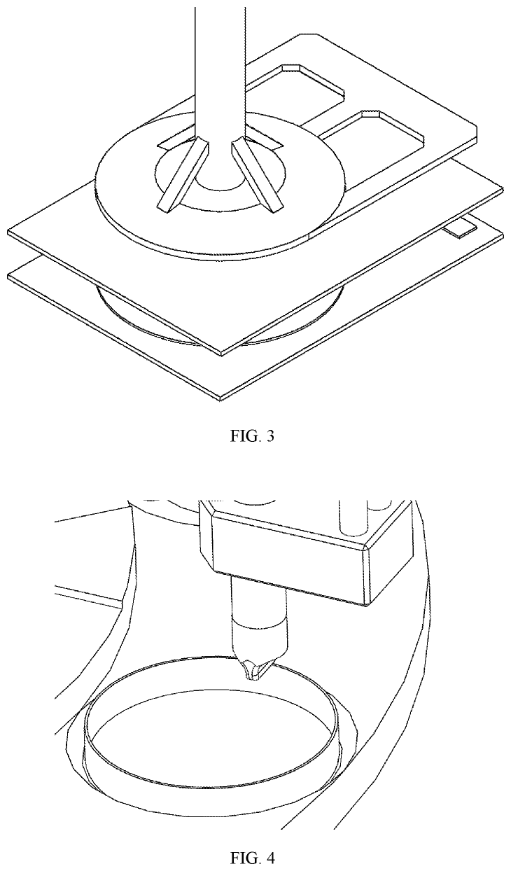

With reference to FIG. 2, shows sample and media (not shown) being simultaneously dispensed into the test area 110 of the microfilm backing card 102 where the sample is dispensed through a pipette tip 114, and the media is dispensed through a nozzle 116. In this example, dispensing of the sample and media is at an assembly position 200, which may be the same or different from assembly position 100.

Subsequently, as shown in FIG. 3, a top film 118 is placed over and onto the deposited sample/media (not shown). In this example, placement of the top film 118 is at an assembly position 300, which may be the same or different from assembly position 100 and/or 200. The top film 118 may be made of plastic or non-plastic-based material(s), and may or may not have a coating on one side (e.g., on the bottom side that contacts the sample/media) of a dry gelling agent(s) with/without dry media or media components (e.g., that may consist of nutrients, buffers, chromogens, pH indicators, selective agents, growth factors, etc.). In a next step, also as shown in FIG. 3 the top film 118 is stamped with stamp 120, the edge 122 of the stamp 120, in combination with the deposited ring 108 defining an area over which the sample will be spread. In this example, stamping with stamp 120 is also at assembly position 300, which may be the same or different from assembly position 100 and/or 200. Alternatively, stamping may be at a different position than placement of the top film 118. The process of stamping spreads and further mixes the sample over the entire test area 110 surface and also will allow the deposited adhesive (e.g., glue), gel, wax, or grease (in this instance, pressure sensitive glue ring 108) to form a barrier, in combination with the backing card 102 and top film 118, to prevent the aqueous sample from escaping the test area 110. Any gelling agent(s), when present on the top film 118 surface, will absorb the applied sample and media mix and the resulting gel allows for trapping any gas that may be generated as the result of microbial growth, where the gas can be of diagnostic value. The deposited linear ribbon 112 of pressure sensitive glue provides an additional anchor for sealing or resealing the top film 116.

The assembled microfilms are labeled (coded in conformity with the sample), and directed/sent or moved into an integrated or non-integrated incubator at an appropriate temperature for each test.

Petri dishes. An alternate exemplary culture device embodiment comprises in situ assembled Petri dishes. For quantitative assessment using Petri dishes, for example, vessels containing extracted samples, or dilution vessels, are moved into a position (e.g., plating position) of the integrated automated system where an appropriate amount of each sample/dilution is plated on a respective Petri dish that is manufactured in situ in a separate module (Petri dish assembly module) of the same automated system.

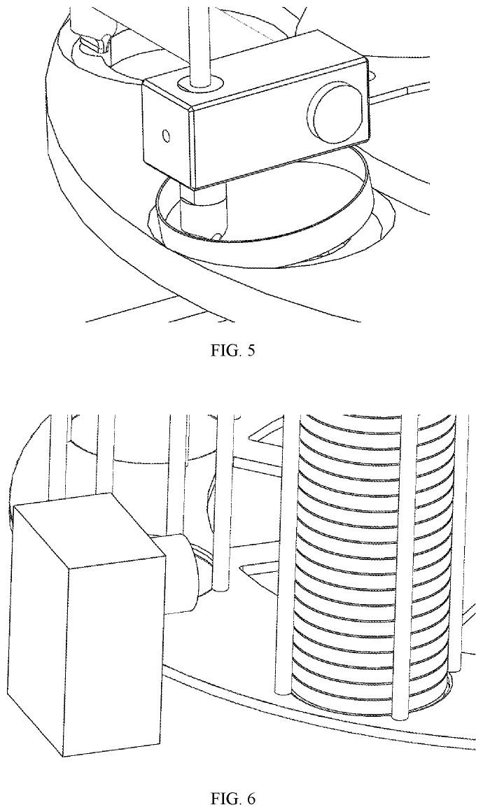

As shown in FIG. 4, based, e.g., on the LIMS instructions, pre-loaded Petri dishes (base 402 plus lid (not shown)) are moved into a Petri dish assembly position 400, where lids (not shown) are removed. A sterilized base media (not shown) containing polymers that congeal in the presence of one or more of cations, gums, water, and appropriate salts containing the cations, etc., is added, via nozzle 404, to cover the inner base surface 406 of the base 402 (e.g., a hot pectin mixture may be used, along with Ca⁺²). Removal of the lid and addition of the sterilized base media may both occur at position 400, or base media addition may occur at a different position within the Petri dish assembly module.

As shown in FIG. 5, the Petri dish base 402 is then tilted at a sufficient angle and for a sufficient time to drain and pool excess (non-adhered) gel, and while tilted, an aspirator nozzle 408 aspirates the pooled gel, leaving a thin, formed gel coating (not shown) on the Petri dish base 402 (a base gel layer). In this example, tilting and aspirating are both performed at an assembly position 400, but could alternatively be performed at different assembly positions.

As shown in FIG. 6, multiple Petri dish bases 402 containing adhered base gel layers may be stacked on an integrated carousel 600 for rapid cooling.

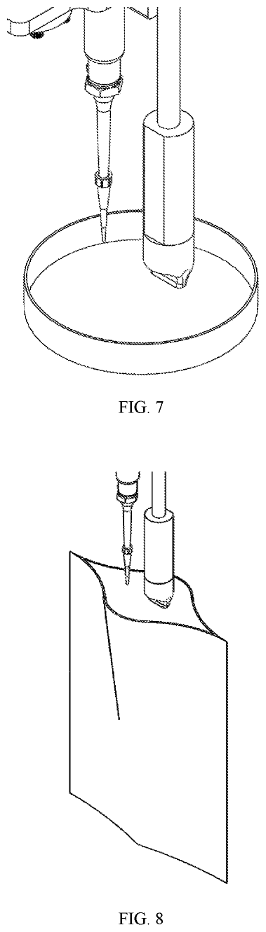

As shown in FIG. 7, the dish base 402 is then moved into another assembly position 700 where an appropriate amount of the sample/dilution is added, via pipette tip 502, on top of the base gel layer, simultaneously with addition, via nozzle 504, of an appropriate amount of a sterile mixture containing one or more medias plus one or more congealable polymers and/or gums that does not congeal at ambient temperature in the absence of the one or more cations (e.g., pectin may be used, which will congeal at ambient temperature in the presence of suitable cations, e.g., Ca⁺²). The one or more cations (e.g., Ca⁺²) are diffusible from the base gel layer into the introduced sterile mixture and the sample to provide for e.g., pectin gel formation over the base gel layer. The Petri dish lid (not shown) is then placed on the dish base 402, the assembled dishes labeled (coded with respect to the sample), gently mixed, and then moved into an incubation stack, which is then moved into an incubator (preferably an integrated incubator) at a specified temperature and for a specified time suitable to promote the growth of colonies corresponding to one or more target microorganisms.

Petri pouches. An additional alternate exemplary culture device embodiment comprises is situ assembled Petri pouches. For quantitative assessment using Petri pouches, vessels containing extracted samples, or dilution vessels, are moved into a position (e.g., pouch plating position) of the integrated automated system where an appropriate amount of each sample/dilution is plated using a Petri pouch that is assembled in situ in a separate module (Petri pouch assembly module) of the same automated system.

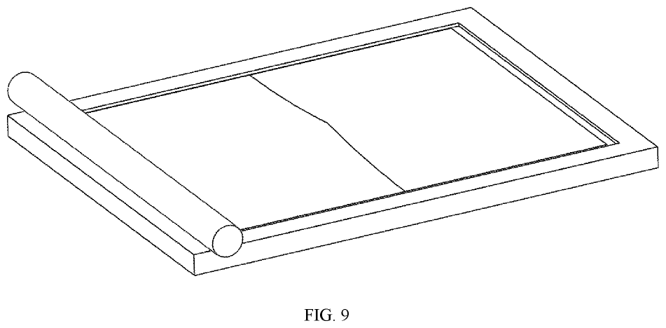

As shown in FIG. 8, based e.g., on the LIMS instructions, pre-loaded Petri pouches 802 (e.g., plastic bags of appropriate size with or without closure mechanism(s)) are moved into a Petri pouch assembly position 800, where the pouch 802 is opened, or is configured with a closable opening, in either case allowing for introduction, via the opening, of a powder or liquid/paste media containing a gelling agent, and introduction of a sample/dilution for inoculation. For inoculation, the sample/dilution may be added to the pouch 802 before, after or simultaneously with the media and/or the gelling agent. Introduction of the sample may be via pipette tip 804, and introduction of the media may be via nozzle 806. After sample inoculation, the petri pouch 802 may be closed (or not), gently mixed, labeled, and, as shown in FIG. 9, passed through a roller 900 or two-plated, in any case with an appropriate gap to distribute the liquid, followed by closing the pouch 802 if not closed prior to mixing the sample and media within the pouch, in either case with subsequent gelation. The pouch 802 is then moved into an incubator (e.g., in a first in, first out manner), preferably an integrated incubator, at a specified temperature and for a specified time suitable to promote the growth of colonies corresponding to one or more target microorganisms.

Incubators. With respect to any culture device embodiments, including those discussed above, processed in the integrated automated system, the incubators may or may not be integrated into the integrated automated system. Preferably using an integrated temperature-controlled incubation chamber, inoculated culture devices are placed and incubated at a temperature suitable to promote the growth of selective microorganisms (e.g., colonies of target organisms). The volume of the incubation chamber is preferably sufficient to incubate a production run of devices (e.g., accumulated production of devices accumulated in a 24 h production run). Alternatively, inoculated culture devices are transferred to an external chamber for ex situ incubation.

Reader modules. After appropriate incubation (e.g., determined by each of the LIMS test protocols), culture devices (e.g., microfilms, Petri dishes, Petri pouches, etc.) are retrieved from the incubators and are moved into the reader module (e.g., comprising an integrated imaging device and an image analysis program) where each culture device (e.g., micro film) is read and the data is transmitted into the e.g., LIMS system. After reading, culture devices are moved into storage devices (integrated or non-integrated) and stored until completion of QC and issuance of reports, after which they are disposed of in an appropriate manner. For example, the color and/or gas production of colonies to be enumerated are analyzed and reported into LIMS system. The incubated culture device may be positioned by a robotic arm, or other suitable appliance, on a first-in-first-out basis from the incubation chamber. In versions of the system where the inoculated device is incubated in an external (non-integrated) chamber, the positioning the culture device for image analysis is programmed accordingly.

Sterilization. Optimally, the system is fully enclosed and under positive HEPA filtered air pressure. All ingredients, media, buffers, microfilm cards, components, tubing, tips, etc., are pre-sterilized before being loaded into the automated system. The automated system may have an integrated clean in place (CIP) system for tubing components, UV lights may also be deployed to maintain a sterile environment (e.g., before starting the unit and during the sanitation cycle).

Stations modules. The integrated automated system includes a plurality of functional modules or stations. The integrated automated system may, for example, include a media preparation portion/station, a sample extracting and diluting portion/station, a media and sample positioning and mixing portion/station, a culture device assembling portion/station, an integrated or non-integrated temperature-controlled incubation chamber, and an image analysis-based colony counting portion/station (e.g., digital) and/or a detection station for qualitative detection of target microbes, or of other analytes.

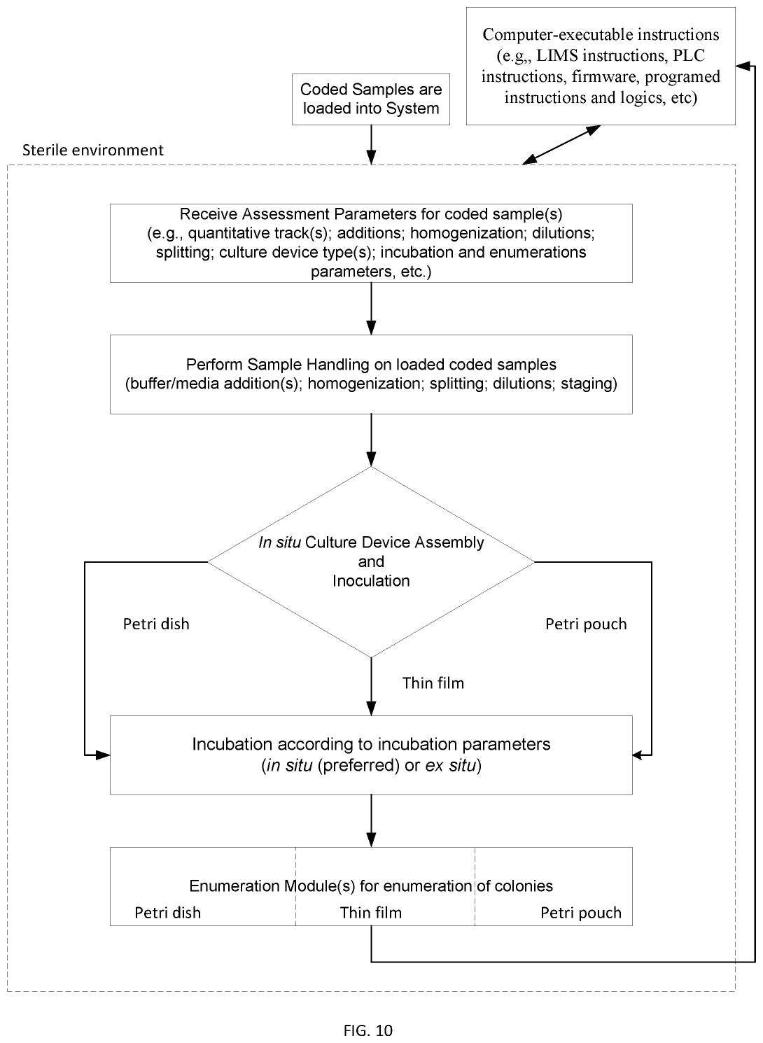

Quantitative tracks. FIG. 10 shows an exemplary high level flow diagram showing implementation of computer-executable instructions for sample processing and quantitative analysis within an automated, integrated laboratory system for quantitative assessment of microbiological and other samples.

For quantitative application tracks, the automated system may, for example, using the functional modules or stations, quantitatively process a sample in the following exemplary steps: (1) a disinfected, dry powder mix of media is solubilized in water and added (optionally along with a gelling agent) to one or more specified version(s) of the in situ assembled microbiological culture devices: e.g., microfilm cards, petri pouch, or petri dishes; (2) a coded sample is extracted, diluted if specified, and added to the specified culture device(s) (e.g., Petri dishes, microfilms and/or Petri pouches, etc.) with attribution, and where the addition of sample or dilution thereof may optionally be simultaneous with media addition to facilitate mixing; (3) for microfilm card versions, a thin-film cover, optionally coated with a gelling agent, may be assembled on the upper surface of the base film (base or bottom card); (4) the assembled inoculated culture devices is/are incubated in a temperature-controlled chamber (incubator) for a specified period of time; (5) microbiological colonies forming on the culture device are analyzed; preferably by an integrated digital image-based, smart recognition and counting module/program, which reads and reports/stores the results, e.g., to LIMS components of the automated integrated system.
Example 1 (herein below) describes a quantitative track of the automated integrated systems, wherein microfilm cards serve as the culture devices, which are assembled in situ and inoculated in situ within a sterile environment for microbiological quantification.
Example 2 (herein below) describes a quantitative track of the automated integrated systems, wherein Petri pouches serve as the culture devices, which are assembled in situ and inoculated in situ within a sterile environment for microbiological quantification.
Example 3 (herein below) describes a quantitative track of the automated integrated systems, wherein Petri dishes serve as the culture devices, which are assembled in situ and inoculated in situ within a sterile environment for microbiological quantification.

Qualitative tracks. In microbiology labs some test samples (e.g., food or non-food sample, etc.) are not only subjected to quantitative determination of the number (enumeration) of various groups of microbes (e.g., yeast and molds, coliforms, fecal coliforms, generic E. coli, Enterobacteriaces, Lactic acid bacteria, Staphylococcus, B. cerus, etc.), but are also, or alternatively subjected to qualitative pathogen and/or spoilage organism detection(s) (qualitative microbiology). Additional qualitative tracks include, but are not limited to genetic testing for the presence of specific genes/genetic markers (e.g., authentication, GMO testing, allergen testing, species identification, ingredient identification, spoilage profile, etc.).

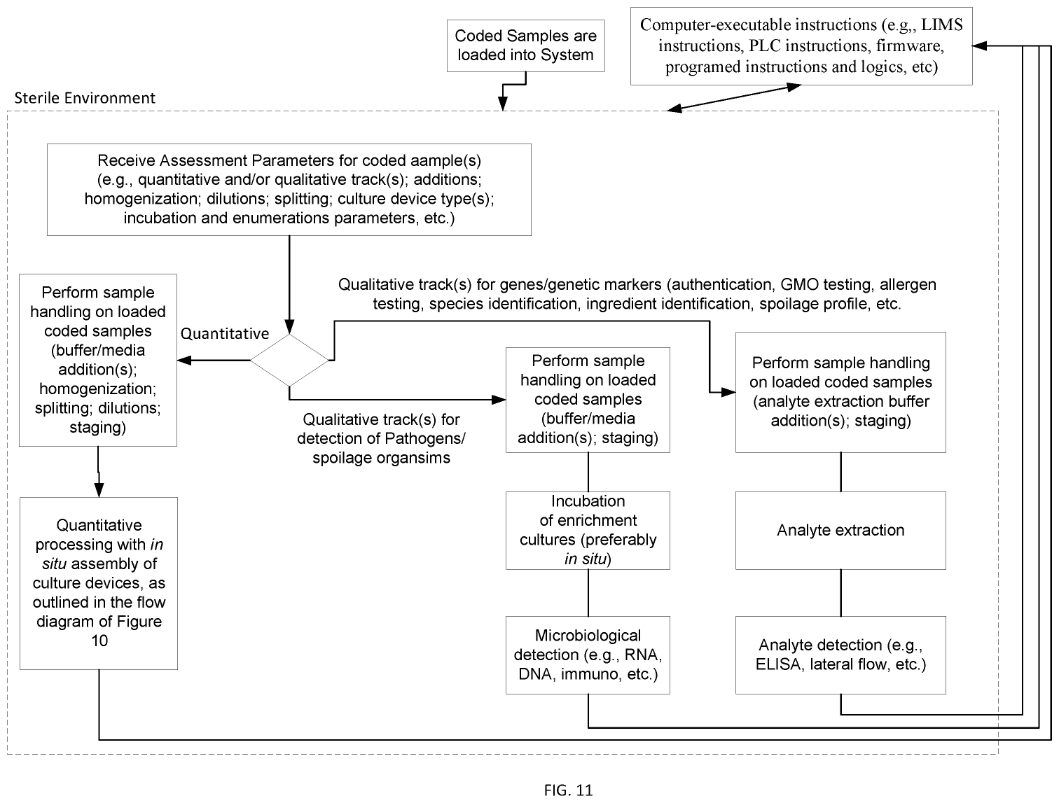

FIG. 11 shows an exemplary high level flow diagram showing implementation of computer-executable instructions for sample processing and quantitative and/or qualitative analysis within an exemplary automated, integrated laboratory system for quantitative and/or qualitative assessment of microbiological and other samples.

For qualitative application tracks involving detection of pathogens or spoilage organisms, for example, the automated systems may, using the functional modules or stations, qualitatively process a sample in the following exemplary steps: (1) concentrated media is added to a coded sample or extraction thereof to provide an enrichment culture with attribution (if multiple analyses are specified, and the medias are not compatible, the sample/extraction may be appropriately split to provide more than one attributed enrichment cultures); (2) the enrichment cultures are directed/sent to an incubator (preferably an integrated incubator) and incubated for a specified time to provide attributed enriched cultures; (3) the attributed enriched cultures are directed/moved to an detection module (preferably an integrated detection module); (4) an aliquot (typically small) of each coded enriched culture is taken and depending on the detection method, samples are transferred into respective, attributed reagent tubes/plates; and (5) detection assays (e.g., nucleic acid (e.g., DNA, RNA) or immunochemical based detection assays) are performed on the contents of the attributed reagent tubes, and the results reported/stored, e.g., to LIMS components of the automated integrated system.

For qualitative tracks involving genetic testing for the presence of specific genes/genetic markers, etc. (e.g., authentication, GMO testing, allergen testing, species identification, ingredient identification, spoilage profile, etc.), the automated systems may, using the functional modules or stations, qualitatively process a sample in the following exemplary steps: (1) a portion of the homogenized sample is diluted with an appropriate analyte extraction buffer; (2) the diluted samples are then directed/transferred to an appropriate extraction module of the system depending on the analyte (e.g., transferred to a bead beating module/components in the case of nucleic acid extraction; to a shaking heating module/components in the case of polypeptide/protein antigen extraction; etc.) for analyte extraction; (3) a portion of the extracted analyte is then directed/transferred to an appropriate detection module of the system (e.g., amplification (e.g., transferred to PCR/Isothermal) module/components in the case of nucleic acids; to ELISA and/or lateral flow module/components in the case of polypeptide/protein antigens; etc.), and the results reported/stored, e.g., to LIMS components of the automated integrated system.

Example 4 (herein below) describes automated integrated microbiological laboratory system embodiments having quantitative (e.g., pathogen or spoilage organism quantification) and/or qualitative tracks, for example, qualitative tracks such as genetic testing for the presence of specific genes/genetic markers (e.g., authentication, GMO testing, allergen testing, species identification, ingredient identification, spoilage profile, etc.).

The automated and integrated systems and processes addresses an unmet need by producing and assembling culture devices (e.g., Petri dishes, microfilms and Petri pouches, etc.) in situ on demand, commensurate with sample testing and quantification in an integrated onsite system, thereby bypassing the need of fabricating the devices at a non-integrated and/or distant site, sending them to irradiation, further sending them to the assay site, and storing them prior to use at the assay site, etc. Rather, sterilized components are assembled in a sterile integrated environment, eliminating the need to further sterilize, ship and store and/or open ex situ fabricated culture devices e prior to inoculation thereof. In combined qualitative/quantitative aspects, the integrated system provides for integrated incubation (for qualitative testing) and/or quantifying (e.g., colony counting) using the in situ assembled culture devices.

### EXAMPLES

The following non-limiting working examples are provided to further illustrate particular embodiments of the invention disclosed herein.

### Example 1

### (Microfilm Cards are Assembled In Situ for Use in an Automated Integrated System as Described Herein)

Particular aspects of the present invention provide an integrated automated system comprising sterile media and/or buffer reagents, culture device parts, disposable and/or permanent dispensing tips, and automated components for media and/or buffer handling, test sample handling, culture device assembly, and microbial enumeration and/or detection, all within a sterile environment; and one or more processors; and memory, including computer-executable instructions (e.g., LIMS instructions, PLC instructions, firmware and programed instructions and logics) that, if executed by the one or more processors, cause the integrated automated system (e.g., upon loading of appropriate quantities of each sample, in an appropriate vessel, bearing appropriate coded instructions (bar codes, etc.)) to perform automated steps of the following method:

Microfilm cards. Particular exemplary microfilm card embodiments may comprise one or more layers of bottom backing card(s), an optional mid-restrictive layer, and a top cover optionally coated with a gelling agent thin-film. For example, in the automated integrated systems described herein, and using sterile ingredients, dry media is dissolved in water. A macromolecular-based thickener may be added to adjust the flow behavior of the media solution. The media solution is applied (may be printed) to a center reservoir of the backing card (based film), which may, as described above, have a layer of e.g., adhesive (e.g., pressure sensitive glue), gel, wax or grease deposited in a geometrical shape defining the outer borders of a test area that will contain the media. The extracted and diluted (where specified) coded sample suspension is dispensed into the center reservoir of base film. The media and sample may be introduced consecutively or simultaneously. A thin-film top cover may be pre-cut and stacked in the integrated automated system. The thin-film top cover may be pre-coated on one side (e.g., the bottom side) with an adhesive and/or a gelling agent powder with/without dry media or media components (e.g., that may consist of nutrients, buffers, chromogens, pH indicators, selective agents, growth factors, etc.). A robotic arm picks the coated thin-film cover and assembles it with the inoculated base film. The assembly is then stamped to distribute the sample over a test area between the base film and the thin-film cover, an attribution label is applied, and the assembled, inoculated coded thin-films are sent into an incubator, preferably an integrated incubator, set at an appropriate temperature and for a specified incubation time.

### Example 2

### (Petri Pouches are Assembled In Situ for Use in an Automated Integrated System as Described Herein)

Particular aspects of the present invention provide an integrated automated system comprising sterile media and/or buffer reagents, culture device parts, disposable and/or permanent dispensing tips, and automated components for media and/or buffer handling, test sample handling, culture device assembly, and microbial enumeration and/or detection, all within a sterile environment; and one or more processors; and memory, including computer-executable instructions (e.g., LIMS instructions, PLC instructions, firmware and programed instructions and logics) that, if executed by the one or more processors, cause the integrated automated system (e.g., upon loading of appropriate quantities of each sample, in an appropriate vessel, bearing appropriate coded instructions (bar codes, etc.)) to perform automated steps of the following method:

Petri pouches. An alternate exemplary culture device embodiment comprises a plastic bag of appropriate size with or without closure mechanism(s). For example, in the automated integrated system as described herein, such culture bags are advanced to an assembly position. A positioned bag is opened, or is configured with a closable opening, in either case allowing for introduction of a powder, or liquid/paste media containing a suitable gelling agent, and introduction of a sample/dilution. After sample inoculation, the petri pouches are closed, gently mixed, labeled, and passed through a roller or two-plated, in any case with an appropriate gap to distribute the liquid and close the pouch (if not closed prior to mixing the sample and media within the pouch), they are then moved into an incubator, preferably an integrated incubator (e.g., in a first in, first out manner).

### Example 3

### (Petri Dishes are Assembled In Situ and Used in an Automated Integrated System as Described Herein)

Particular aspects of the present invention provide an integrated automated system comprising sterile media and/or buffer reagents, culture device parts, disposable and/or permanent dispensing tips, and automated components for media and/or buffer handling, test sample handling, culture device assembly, and microbial enumeration and/or detection, all within a sterile environment; and one or more processors; and memory, including computer-executable instructions (e.g., LIMS instructions, PLC instructions, firmware and programed instructions and logics) that, if executed by the one or more processors, cause the integrated automated system (e.g., upon loading of appropriate quantities of each sample, in an appropriate vessel, bearing appropriate coded instructions (bar codes, etc.)) to perform automated steps of the following method:

Petri dishes. Additional alternate culture devices embodiment comprise Petri dishes. For example, in the automated integrated systems as described herein, Petri dishes are pre-loaded in the system. The dishes move into an assembly position, where lids are removed. A sterilized base media containing polymers that congeal in the presence of one or more of cations, gums, water, and appropriate salts containing the cations, etc., is added to cover the base to provide a thin formed gel coating on the base of the Petri dish (a base gel layer). The plate is then moved into another assembly position where an appropriate amount of the sample is simultaneously added, on top of the base gel layer, with an appropriate amount of a sterile mixture containing one or more medias plus one or more congealable polymers (e.g., pectin) and/or gums that does not congeal at ambient temperature in the absence of the one or more cations (e.g., Ca⁺²). The one or more cations are diffusible from the base gel layer into the introduced sterile mixture and the sample to provide for gel formation over the base gel layer. The lid is then placed on the plates, the plates labeled (coded with respect to the sample), and gently mixed and moved into an incubation stack which is then moved into an incubator (preferably an integrated incubator) at a specified temperature and for a specified time suitable to promote the growth of colonies corresponding to one or more target microorganisms.

### Example 4

### (An Automated Integrated Microbiological Laboratory for Quantitative and Qualitative Microbiology, and Other Qualitative Analysis is Provided)

Overview. In microbiology labs some test samples are subjected to qualitative microbiology (e.g., pathogen detection(s), etc.), as well as quantitative determination of number of various groups of microbes such as yeast and molds, coliforms, fecal coliforms, generic E. coli, Enterobacteriaces, Lactic acid bacteria, Staphylococcus, B. cerus, etc.

Particular aspects of the present invention provide an integrated automated system for quantitative and/or qualitative microbiology (or other qualitative analysis of samples), comprising sterile media and/or buffer reagents, culture device parts, disposable and/or permanent dispensing tips, and automated components for media and/or buffer handling, test sample handling, culture device assembly, and microbial enumeration and/or microbial detection and/or detection of other analytes, all within a sterile environment; and one or more processors; and memory, including computer-executable instructions (e.g., LIMS instructions, PLC instructions, firmware and programed instructions and logics) that, if executed by the one or more processors, cause the integrated automated system (e.g., upon loading of appropriate quantities of each sample, in an appropriate vessel, bearing appropriate coded instructions (bar codes, etc.)) to perform automated steps of the following exemplary method(s):
A: Quantitative and Qualitative Analysis Tracks
   1. Sample collection device: e.g., stomacher bags or wide mouth jars;
   2. Sample arrival and registry: Sample submission forms are electronically received. Samples are barcoded/have a unique identifying code;
   3. Samples are loaded onto sample holding trays and are fed into the machine;
   4. LIMS loads the analysis request into the automated system as each sample identifier is read;
   5. Sample container is e.g., decapped or sample bag is opened, an appropriate buffer is to each added, based on the weight of the sample, collection device is closed and sample goes through the homogenizer;
   6. Sample homogenized;
   7. For quantitative analysis, the sample container is decapped/opened, a portion of the sample is taken for dilution (e.g., serially and/or parallel for quantitative test, that is moved into the quantitative track using in situ assembled culture devices as discussed elsewhere herein in detail);
   8. For qualitative analysis, portions of the initial homogenized samples, in appropriate vessels, are be directed to the qualitative modules/tracks (e.g., pathogen, spoilage organisms, etc.).
B. Pathogen or Spoilage Organism Track(s)
   B1. For following a qualitative track such as a pathogen or spoilage organism track, concentrated media is added to the homogenized sample portions for enrichment, and if more than one analysis is desired for a sample, and the required medias are not compatible, the sample will be split (e.g., into two) and appropriate concentrated media added to each;
   B2. After concentrated media is added, the enrichment sample vessels(s) are directed/moved towards incubators (incubation module) and to follow a qualitative e.g., pathogen or spoilage organism track;
   B3. Enrichment sample vessels are placed into appropriate incubators, and the samples incubated at an appropriate temperature (e.g., range of from 25-45° C.) for an appropriate time (e.g., range of from 4-48 hours);
   B4. At the end of the incubation period, enriched sample(s) are automatically transferred to the testing/detection module;
   B5. The sample vessels are then opened, and an appropriate amount of the enriched sample is taken for detection by an appropriate selected detection method (e.g., nucleic acid (e.g., DNA/RNA); immunochemistry, etc.). For example, for a nucleic acid based detection method, appropriate enriched sample aliquots is transferred into attributed reagent tubes/plates for nucleic acid detection assays (e.g., nucleic acid (e.g., into DNA/RNA) extraction tubes). Reagents are then added, the tubes mixed and placed on a magnetic separation module, where the supernatant is removed, followed by a wash, magnetic separation, and supernatant removal (which steps may be repeated). After the final wash, an appropriate buffer is added bring/extract the nucleic acids into solution. A portion of the extract is then transferred to an amplification tube containing an appropriate amplification reaction buffer and reagents. The amplification tube with buffer and reagents, is then transferred to the PCR/Isothermal amplification module of the system, which conducts and reads the amplification reaction, and reports the results, e.g., to LIMS components of the automated integrated system. In alternate embodiments, an appropriate amount of an enriched sample may be directly added to the amplification reaction tube, followed by placement into the amplification module (PCR/Isothermal), etc.
C. Genetic Testing Tracks
   C1. For following a qualitative track such as a genetic testing for the presence of specific genes/genetic markers, etc. (e.g., authentication, GMO testing, allergen testing, species identification, ingredient identification, spoilage profile, etc.), appropriate assays/testing is implemented by the integrated automated system. For example, testing based on use of magnetic beads and amplification may be implemented.
   C2. In such a testing approach, for example, a portion of the homogenized sample is diluted in a sample tube with extraction buffer and the mixture transferred to the bead beating module/component of the integrated system, where coated magnetic beads are added to the mixture in the tube. The diluted sample and beads are then mixed in the tube, the tube placed on a magnet, and the supernatant removed. A wash buffer is then added to wash the beads, the tube placed on the magnet, and the supernatant removed, after which the beads are suspended in an appropriate volume of a buffer. For each such test sample preparation, approximately 1-5 microliters of the suspended beads is transferred to a respective appropriate amplification tube containing an appropriate amplification reaction buffer and reagents. The amplification tube with buffer and reagents, is then transferred to the PCR/Isothermal amplification module of the system, which conducts and reads the amplification reaction, and reports the results, e.g., to LIMS components of the automated integrated system.
   C3. For tests that require immunological testing, for example, appropriate extraction buffers are added to portions of the original homogenized samples or dilutions thereof in sample tubes, which are then directed to a shaking heating/module component of the integrated automated system. After an appropriate residence in the shaking heating module, portions of each sample are transferred to a analysis module of the system having ELISA plates and/or lateral flow device components, followed by automated addition of reagents, incubations, washes, and reading and reporting the results, e.g., to LIMS components of the automated integrated system.

What is claimed is:

1. A system for qualitative and/or quantitative microbiological assessment, comprising:
an integrated automated system comprising sterile media and/or buffer reagents, culture device parts, and automated components for media and/or buffer handling, test sample handling, culture device assembly, and microbial enumeration and/or detection, all within a sterile environment; and
one or more processors; and memory, including computer-executable instructions (e.g., LIMS instructions) that, if executed by the one or more processors, cause the integrated automated system to determine that a coded test sample, loaded into the system, is to be quantitatively and/or qualitatively assessed, and
for quantitative assessment,
a) assemble, in situ, using the culture device parts, a specified type of microbiological culture device for culturing and quantitatively processing the coded test sample;
b) inoculate the specified type of culture device, as part of (e.g., during or after) the in situ assembly, with an appropriate amount of the coded test sample, or an extraction and/or dilution thereof; and
c) attribute the inoculated culture device to the coded test sample, wherein the assembling and inoculating are performed in situ by the integrated automated components of the system in the sterile environment, thereby eliminating the need to sterilize, ship, store, and/or open ex situ fabricated culture devices prior to inoculation thereof; and/or
for qualitative assessment,
A) add an amount of a media or concentrated media into at least a portion of the coded test sample or extraction thereof, to provide a coded enrichment culture; and/or
A1) dilute a portion of the coded test sample or extraction thereof by adding an amount of a suitable analyte extraction buffer.

2. The system of claim 1, wherein execution of the computer-executable instructions further causes the system to:
for quantitative assessment,
d) direct the inoculated, attributed culture device for incubation at a temperature and for a time suitable to promote the growth of colonies corresponding to one or more target microorganisms;
e) enumerate the colonies using the enumeration components in situ; and
f) store and/or transmit the enumeration data attributed to the coded test sample; and/or
for qualitative assessment, execution of the computer-executable instructions further causes the system to:
B) direct the enrichment culture for incubation in an incubator at a temperature and for a time suitable to promote the growth of one or more target microorganisms to reach detectable levels (and preferably become uniform throughout) to provide an enriched sample;
C) detect, using at least an aliquot of the enriched sample, the one or more target microorganisms using the detection components in situ; and
D) store and/or transmit the detection data attributed to the coded test sample for storage and/or reporting and/or analysis; and/or
B1) direct the analyte sample of A1) to a suitable analyte extraction module/components for analyte extraction, using extraction components of the system;
C1) detect, using a portion of the extracted analyte of B1) in a detection module/component of the system one of more extracted analytes; and
D1) store and/or transmit the detection data of C1) attributed to the coded test sample for storage and/or reporting and/or analysis.

3. The system of claim 2, wherein directing, in d), the inoculated, attributed culture device for incubation, and/or directing, in B), the enrichment culture for incubation, comprises incubating in situ using an incubator integrated into the system, and/or incubating ex situ using a non-integrated incubator.

4. The system of claim 2, wherein:
enumerating, in e), the colonies in situ comprises using an integrated imaging device and image analysis program; and/or
detecting, in C), the one or more target organisms in situ comprises using a suitable detection assay in situ; and/or
detecting in C1), the one or more extracted analytes in situ comprises using a suitable detection assay in situ.

5. The system of claim 4, wherein the detection assay in C) is based on one or more selected from RNA, DNA, and immunochemistry; and/or
wherein the detection assay in C1) is based on one or more selected from ELISA and/or lateral flow.

6. The system of claim 1, wherein:
for quantitative assessment,
when the coded test sample loaded into the system is a primary sample, execution of the computer-executable instructions further causes the system to:
mix or homogenize the coded test sample with a suitable amount of a specified buffer, and/or media, to provide an extracted sample;
withdraw a subportion of the extracted sample; and
dilute, if so specified, the withdrawn portion to provide the extracted sample or the dilution(s) thereof for inoculation of the assembled culture device; and/or
when the coded test sample loaded into the system is an extracted sample, execution of the computer-executable instructions further causes the system to:
withdraw a subportion of the extracted sample; and
dilute, if so specified, the withdrawn portion to provide the extracted sample or the dilution(s) thereof for inoculation of the assembled culture device; and/or
for qualitative assessment,
when the coded test sample loaded into the system is a primary sample, execution of the computer-executable instructions further causes the system to:
prior to A), mix or homogenize the coded test sample with a suitable amount of a specified buffer to provide an extracted sample.

7. The system of claim 1, wherein the computer-executable instructions comprise one or more of LIMS instructions, PLC instructions, firmware, and programed instructions and logics.

8. The system of claim 1, wherein, for quantitative assessment, the specified type of in situ-assembled microbiological culture device comprises at least one selected from the group consisting of microfilm card, Petri-dish containing gel and or gum based media, and Petri-pouch.

9. The system of claim 8, wherein the in situ-assembled microbiological culture device is a Petri-dish, and wherein for the in situ assembly, execution of the computer-executable instructions further causes the system to:
remove the lid of a pre-loaded Petri dish having a base and a lid;
introduce an appropriate amount of a sterile mixture containing one or more medias plus one or more congealable polymers and/or gums;
introduce an appropriate amount of the coded test sample, or the extraction and/or the dilution thereof;
place the lid on the base of the Petri dish; and
mix the introduced sterile mixture and the sample, extraction or dilution thereof prior to gelation.

10. The system of claim 9, wherein the introducing of the sterile mixture and the introducing of the coded test sample, or the extraction and/or the dilution thereof is simultaneous.

11. The system of claim 9, wherein the one or more congealable polymers and/or gums of the introduced sterilized mixture does not congeal at ambient temperature in the absence of one or more cations, and wherein, prior to introducing the sterile mixture and the sample, extraction or dilution thereof, execution of the computer-executable instructions further causes the system to:
cover the base with a base gel layer containing the one or more cations, which are diffusible from the base gel layer into the introduced sterile mixture and the sample, extraction or dilution thereof.

12. The system of claim 11, wherein the one or more congealable polymers and/or gums of the introduced sterile mixture comprise pectin and/or alginate, and wherein the one or cations of the base gel layer comprises a divalent or trivalent cation.

13. The system of claim 11, wherein the base gel layer comprises agar, gelatin, silica gel, or carrageenan.

14. The system of claim 11, wherein the one or cations comprises Ca²⁺.

15. The system of claim 9, wherein the sterile mixture is introduced in liquid form, or wherein the one or more congealable polymers and/or gums of the introduced sterile mixture is initially introduced as a coating in powder form, which upon the introduction of the media in liquid form and/or of the coded test sample, or the extraction and/or the dilution thereof, absorbs and forms a gel over the base gel layer.

16. The system of claim 8, wherein the in situ-assembled microbiological culture device is a microfilm device having a backing card and a top cover, and wherein for the in situ assembly, execution of the computer-executable instructions further causes the system to:
apply sterile media to the surface of the backing card;
introduce an appropriate amount of the coded test sample, or the extraction and/or the dilution thereof to be in contact with the media to form a mixture; and
place a top cover over the backing card; and
stamp the placed top cover to distribute the mixture over a desired test area of the backing card.

17. The system of claim 16, wherein the surface of the backing card defines a reservoir to receive the media and the coded test sample, or the extraction and/or the dilution thereof, the reservoir having a surface area defining the desired test area.

18. The system of claim 16, wherein, before or after applying sterile media to the surface of the backing card, execution of the computer-executable instructions further causes the system to:
apply an adhesive, gel, wax or grease in a pattern to define the desired area over which the mixture is distributed.

19. The system of claim 18, wherein the adhesive comprises a pressure sensitive glue.

20. The system of claim 15, wherein prior to placing the top cover, execution of the computer-executable instructions further causes the system to:
introduce an amount of concentrated media to be diluted by the introduced coded test sample, or the extraction and/or the dilution thereof.

21. The system of claim 18, wherein the adhesive, gel, wax or grease is applied prior to the sterile media.

22. The system of claim 16, wherein the applying of the sterile media to the surface of the backing card comprises:
printing the sterile media onto the surface of the backing card; and/or otherwise dispensing the sterile media onto the surface of the backing card followed by drying.

23. The system of claim 16, wherein the top cover is pre-coated, on a surface, with a gelling agent powder, with or without dry media or media components.

24. The system of claim 8, wherein the in situ-assembled microbiological culture device is a Petri pouch, having or configured to have an opening.

25. The system of claim 24, wherein for the in situ assembly, execution of the computer-executable instructions further causes the system to:
introduce, via the opening, an appropriate amount of a sterile mixture containing one or more medias plus one or more congealable polymers and/or gums;
introduce, via the opening, an appropriate amount of the coded test sample, or the extraction and/or the dilution thereof;
mix the introduced sterile mixture and the sample, extraction or dilution thereof within the pouch;
roll, press or otherwise form the pouch to distribute, prior to gelation, the mixed components to provide a desired gel thickness and surface area; and
close, prior to or after the mixing and/or the rolling, the opening.

26. The system of claim 24, wherein the sterile mixture is introduced in liquid form, or wherein the one or more medias and the one or more congealable polymers and/or gums of the introduced sterile mixture are initially introduced in powder form, which upon the introduction of the coded test sample, or the extraction and/or the dilution thereof, absorbs and forms a gel.

27. The system of claim 25, wherein the one or more congealable polymers and/or gums of the introduced sterilize mixture does not congeal at ambient temperature in the absence of one or more cations, and wherein the inside of the Petri pouches is pretreated with the one or more cations, which are diffusible into the introduced sterile mixture and the sample, extraction or dilution thereof.

28. The system of claim 27, wherein prior to introducing the sterile mixture and the sample, extraction or dilution thereof, execution of the computer-executable instructions further causes the system to:
pretreat the inside of the Petri pouches with the one or more cations, which are subsequently diffusible into the introduced sterile mixture and the sample, extraction or dilution thereof.
